(19) United States
(12) Patent Application Publication
(10) Pub. No.: US 2021/0395696 A1
(43) Pub. Date: Dec. 23, 2021

(54) ISOLATED NASOPHARYNGEAL CARCINOMA CELLS AND DERIVATIVES PREPARED THEREOF

(71) Applicant: INSTITUTE FOR MEDICAL RESEARCH, Kuala Lumpur (MY)

(72) Inventors: Alan Soo Beng KHOO, Kuala Lumpur (MY); Norazlin ABDUL AZIZ, Kuala Lumpur (MY); Sin Yeang TEOW, Kuala Lumpur (MY); Mohd Firdaus CHE MAT, Kuala Lumpur (MY); Marini MARZUKI, Kuala Lumpur (MY); Tai Lin CHU, Kuala Lumpur (MY); Munirah AHMAD, Kuala Lumpur (MY)

(73) Assignee: INSTITUTE FOR MEDICAL RESEARCH, Kuala Lumpur (MY)

(21) Appl. No.: 17/289,132

(22) PCT Filed: May 29, 2019
(86) PCT No.: PCT/MY2019/000019

§ 371 Date: Apr. 27, 2021

(30) Foreign Application Priority Data
Jul. 17, 2018 (MY) PI 2018702497

(51) Int. Cl. C12N 5/09; A01K 67/027; C12N 5/071; G01N 33/50

(52) U.S. Cl. CPC C12N 5/0693; C12N 2513/00; C12N 5/0632; G01N 33/5008; A01K 2207/12; A01K 2227/105; A01K 2267/0331; C12N 2500/25; C12N 2501/11; C12N 2501/115; C12N 2501/39; C12N 2501/727; C12N 2503/02; C12N 2510/00; A01K 67/0271

(57) ABSTRACT

There is disclosed patient derived xenograft (PDXs) cells/systems/models and/or derivatives, parental (unlabelled) and/or labelled, expressing a fluorescent protein or a luciferase, or a combination thereof; for evaluating therapies comprising nasopharyngeal carcinoma (EBV positive and/or EBV negative). In another embodiment, there is disclosed a method of evaluating the efficacy of an agent used to treat nasopharyngeal carcinoma (NPC) comprising: preparing a non-human model; whereby the non-human model carries cells from NPC xenograft; labelling the cells from the NPC xenograft with gfp-luc2 marker using a lentiviral vector system; and growing the cells in short term in vitro culture; including adaptation of said culture into multi-well plates for use in further screening and/or evaluation assays; wherein the NPC xenograft is PDX.

### FIELD OF INVENTION

The present invention generally relates to methods of preparing isolated nasopharyngeal carcinoma (NPC) cells and derivatives; and more particularly for assays based on cells from patient-derived NPC xenografts. In one embodiment, the present invention further relates broadly to the use of such cells, derivatives and animal models in the study of cancer, preferably for NPC.

### BACKGROUND

One of the main challenges in developing and screening of target anti-cancer drugs for Nasopharyngeal carcinoma (NPC) is the lack of cell lines for stable and sustainable growth of cancer tissue in cell culture as well as in animal models, which in turn would allow further in vitro and in vivo investigations or studies of the progression of the respective cancer and effects of compounds, chemical entities or drugs on the cancer. NPC is one of the most common malignancies in Southeast Asian region and is also a major cancer particularly in Malaysia. However, research on NPC is impeded primarily by insufficient number of cell lines and xenografts that could serve as an appropriate panel of tumour model to better represent the biology of NPC. Over the recent years, the development of in vitro and in vivo models which simulates the desired biological environment and then recapitulate the biological properties of NPC has proven challenging. Primary or patient-derived NPC samples are difficult to grow in vitro and only a small number of successfully established NPC cell lines recapitulate and maintain the properties of NPC (eg Cheung S T, Huang D P, Hui A B, Lo K W, Ko C W, Tsang Y S, Wong N, Whitney B M, Lee J C. Nasopharyngeal carcinoma cell line (C666-1) consistently harbouring Epstein-Barr virus. Int J Cancer. 1999 Sep. 24; 83(1):121-6). These are inadequate to represent the heterogeneity of NPC.

In view of this, there is an urgent need for reliable alternatives in preparing models for the evaluation, diagnosis and generation of therapies for cancers.

An object of the present invention is to provide a stable supply of NPC cells to be used in evaluation and/or translational studies in nasopharyngeal carcinoma, including studies to develop biomarkers and/or therapies.

### SUMMARY OF INVENTION

In one aspect, the present invention provides a method of preparing stable and serially transplantable nasopharyngeal cancer (NPC) cells comprising: isolating NPC cells from NPC patients and inoculating said cells in an immunocompromised non-human or animal model; harvesting NPC xenograft tissues from said non-human/animal model; subjecting said NPC xenograft tissues to tissue dissociation and digestion process to obtain NPC cells; growing the cells in in vitro culture; including adaptation of said culture into multi-well plates for use in further assays.

Preferably, the method further comprising labelling the cells with gfp-luc2 marker using a lentiviral vector system.

Preferably, the use of lentiviral vector system include the combination of hexadimethrine bromide and spinoculation with viral supernatant concentrate; and re-inoculated into the non-human model after transduction to avoid the need for prolonged in vitro culture and to reduce risk of genetic alterations to the original cells during in vitro culture and for PDX cells which are difficult to culture in vitro.

In a preferred embodiment, the method further includes evaluating the viability of NPC PDX cells grown in multi-well plate format; said evaluation further comprising performing non-lytic luciferase assays of NPC xenograft cells cultured in multi-well plate format for drug screening for NPC; establishment of a non-lytic luciferase assay to measure cell viability in a non-destructive manner which could be used in mono-culture or in co-culture systems.

In another preferred embodiment, the method further includes the step of determining whether the cancer cells in non-human in vivo model or models provides a sufficient representation or duplication in the characteristics of tumours for use in further cancer studies.

Preferably, the method further includes monitoring tumour burden and/or metastasis for NPC non-human in vivo model and evaluating at least one of the following: candidate biomarkers, candidate drug targets, effects of drugs/bioactive compounds, a new chemical entity, candidate drugs, irradiation and/or other therapeutic agents, and studies on gene function, or a combination thereof.

Preferably in any one of the methods described, the cells include EBV positive cells.

In one embodiment, the adapting said cells into multi-well plate format includes into 96-well plate format or 384-well plate format.

In another aspect, the present invention discloses a method of evaluating the efficacy of an agent against NPC comprising: preparing a non-human model; whereby the non-human model carries cells obtained from a patient-derived NPC xenograft; labelling the cells with gfp-luc2 marker using a lentiviral vector system; and adapting/growing the cells in in vitro 2D/3D/ex-vivo culture; including adaptation of said culture into multi-well plate format for use in further screening and/or evaluation assays, in monocuhure or co-culture systems.

In a preferred embodiment, evaluating the efficacy of an agent wherein the in vitro culture comprises: 0-25% heat deactivated calf serum, 0-5× Glutamax, 0-5× of Antibiotic/Antimycotic, 0-5×B-27 Supplement, 0-5× Insulin Transferrin-Selenium-A, 0-5 mg/ml Hydrocortisone, 0-50 μM ROCK Inhibitor, 0-50 ng/ml Epidermal Growth Factor and 0-50 ng/ml Basic Fibroblast Growth Factor.

Further in accordance with an embodiment of the present invention, the method of evaluating the efficacy of an agent described herein, wherein the in vitro culture comprises: 5-10% heat deactivated calf serum, lx Glutamax, lx of Antibiotic/Antimycotic, 1× B-27 Supplement, lx Insulin Transferrin-Selenium-A, 0.5 mg/ml Hydrocortisone, 5-10 μM ROCK Inhibitor, 5-10 ng/mi Epidermal Growth Factor and 5-10 ng/ml Basic Fibroblast Growth Factor.

Preferably, the agent is selected from one of the following: drug, bioactive compound, chemical entity, biological agent, irradiation or a combination thereof.

Preferably, testing or evaluating the efficacy of an agent against NPC includes studying of effects agent in eliminating, killing, and/or slowing the growth of cancer cells.

In one embodiment, at least a cell is EBV-positive.

Further in another aspect, the present invention discloses a non-human in vitro model and/or in vivo model adapted for evaluating efficacy of an agent against NPC comprising patient-derived NPC xenograft cells; said xenograft, parental or expresses at least one type of fluorescent protein and/or a luciferase.

In another aspect, the present invention discloses an NPC-PDX model comprising at least one the following serially transplantable xenografts (PDXs) identified as Xeno-284. Xeno-287 or Xeno-B110, Xeno-G514, Xeno-G517, Xeno-0518, Xeno-G244 with characteristics similar to those as disclosed in the description; and all its derivatives.

In yet a further aspect, the present invention discloses isolated cells obtained from NPC patient-derived serially transplantable xenografts identified either as Xeno-B110, Xeno-284 and Xeno-287, Xeno-GS14, Xeno-0517, Xeno-G518, Xeno-G244 having characteristics similar to those as disclosed in the description and drawings; and all its derivatives.

Preferably, at least one of the xenografts exhibits mutations in nuclear factor-kappa-light-chain enhancer of activated B-cells (NF-κB), phosphatidylinositol 3′-kinase (PI3K) and/or, mitogen-activated protein kinase (MAPK) and/or mismatch repair (MMR) pathways and/or other pathways (i.e. p53) and/or expression of EBV LMP-1.

Preferably, at least one of the xenografts exhibits either one of the following genomic characteristics: CYLD (c.1112C>A, p.Ser371Ter); CYLD (c.1461G>A, p.W487X); NFKB1 (c.574C>T, p.Arg192Trp); PTEN (c.765_776delAGAGTTCTTCCA, p.Glu256_His259del); PIK3CA (c.1633G>A, Glu545Lys); FGFR2 (c.1436G>C. p.Arg479Thr); FGFR2 (c.1411G>C, p.Glu471Gin); TP53 (c.217delG, p.Val73TrpfsTer50); MLH1 (c.1121T>A, p.Leu374Gln); HLA-A (c.3370>T, p.Glu113Tcr); HLA-A*24:02, 03:02, HLA-B*58:01, 13:02; HLA-A*02:07, HLA-B*46:01; HLA-A*11:01, 24:02 HLA-B*15:02, 35:05; HLA-A*24:02, 24:07 HLA-B*15:02, 35:05.

Preferably, at least one of the xenografts described herein is tested EBV positive.

Preferably, the model as described herein can be used in determining tumour progression over time and/or identifying resistance and/or sensitivities of the cancer cells against candidate biomarkers, candidate drug targets, effects of drugs/bioactive compounds, a new chemical entity, candidate drugs, biological agent, irradiation and/or other therapeutic agents, and studies on gene function.

Preferably, the cells as disclosed herein are co-cultured with immune or other stromal cells in an in vitro assay.

In yet another aspect, the present invention discloses a method for labelling NPC cells that are difficult to culture in vitro and without extended in vitro propagation, the method comprises: labelling NPC xenografts with gfp-luc2 marker using a lentiviral vector system in a transduction process; and re-inoculating said xenografts into an animal model prior to selection of transduced cells; harvesting the xenograft established from the re-innoculated cells for selection, then re-innoculating the selected cells into an animal model; harvesting cells from xenograft established from the selected reinnoculated cells for further propagation, analysis or for adaptation into 2D/3D culture.

Preferably, the transduction of lentiviral vector system includes a combination of hexadimethrine bromide and spinoculation with viral supernatant concentrate.

### BRIEF DESCRIPTION OF THE FIGURES

FIG. 1: Overall view of the method of preparing NPC cells for further evaluation in accordance with a preferred embodiment of the present invention;

FIG. 2: A detailed example representing the stages involved in accordance with the method shown in FIG. 1, according to a preferred embodiment of the present invention;

FIG. 3: Histological and immunohistochemical (IHC) characterisation of Epstein-Barr virus (EBV) negative and EBV positive NPC patient derived xenografts (PDXs);

FIG. 4: Examples of experimental results depicting the verification of PDXs origin (human vs mouse) via PCR. HeLa, NIH3T3, Namalwa, Jiyoye and Blank are control samples. Nil=empty lane;

FIG. 5: Detection of human papilloma virus (HPV) in PDXs by PCR using the HPV consensus primers GP5+/GP6, β globin as control, and detection of p16INK4 expression by IHC staining; in accordance with experimental examples of embodiments of the present invention;

FIG. 6: Experimental results depicting further characterization of EBV positive PDXs. (A) EBV genotyping by PCR. Namalwa and Jiyoye cell lines are positive controls for EBV Type I and EBV Type II respectively. (B) IHC staining of latent membrane protein 1 (LMP1) showing G514 PDX highly expressing LMP1, (C) Results from Epstein-Barr encoded RNA in situ hybridization (EBER ISH) staining showing EBV is maintained in early and later passages of the parental as well as gfp-luc2 labelled PDXs;

FIG. 7: Imaging of gfp-luc2-labelled-xenografts. (A) Images of freshly harvested xenograft tumours (bright field (BF) and FITC channel) captured ex vivo using fluorescence stereomicroscope. Images of in vivo tumours (luminescence) were photographed using IVIS optical imaging system. (B) Bright field, FITC and merged images of xenograft tumours that were adapted into short-term 2D- and 3D culture models in vitro. Images were captured using IN-Cell high-content cell analyzer. (C) Luminescence of xenograft cells seeded at different cell number into 96-well plates. Luminescent signal was detected using IVIS optical imaging system. Left panel: XenoB110-gfp-luc2; Right panel: Xeno284-gfp-luc2;

FIG. 8: Characterization of XenoLuc bioluminescence cell-based assay. (A) Diagram showing the basic concept of luciferase/luciferin system. (3) The signal of XenoLuc assay (XenoLight) was compared with the two commercial assays—Cell titer Glo (lytic) and Real time Glo (non-lytic), respectively. (C) The signal stability of assay was compared with Real time Glo. (D) Cytotoxicity of XenoLight was determined by MTS assay. Data is the average of triplicate from three tumours (Xeno-B110-gfp-luc2) and the standard deviation is represented by error bars.

FIG. 9: Example of experimental results showing linear correlation between number of cells (Xeno-B110-gfp-luc2) and luminescence signals when measured using XenoLuc bioluminescence cell-based assay;

FIG. 10: Examples of experimental cell measurement results in relation to the use of an assay in accordance with an embodiment of the present invention. A) Measurement of luminescence in two-fold serially diluted non-depleted xenograft cells. Unlabelled parental xenograft cells and normal human dermal fibroblast (NHDF) cells were used as negative controls. These cells were seeded onto 96-well plates as 2) monolayer culture, and the luminescence was measured after 4 days. (B) Measurement of luminescence of mouse cell-depleted xenograft cells, cultured in 2D and 3D in vitro models.

FIG. 11: Cell proliferation analysis of XenoB110-gfp-luc2 by GFP fluorescence intensity using IN-CELL. Developer software. (A) Non-depleted xenografts; (B) Mouse cell-depleted xenografts; (C) 3D spheroids. The images inset shows the increasing size of spheroids resulting from the increase of seeded cell number;

FIG. 12: Examples of different in vitro adaptation for establishing 2D cell culture from PDXs. (a) Explant to 2D culture, (b) spheroid to 2D culture, (c) single cells to 2D culture;

FIG. 13: Examples of data showing characterization of derivatives from PDXs—in vitro culture in accordance with an embodiment of the present invention (A) PDX cells in 2D and 3D spheroid culture expressing epithelial marker, EPA. (B) Histological characterization of cell derivatives from short-term in vitro culture of PDX. Cell derivatives showed positive expression of pan-CK and EBER. (C) Growth curve of PDX cells in 2D and, (D) 3D spheroid culture;

FIG. 14: Examples of data showing use of the assay to test the effect of different supplements on cell viability. Xenografts cells were seeded onto 96-well and RPMI1640/10% FBS basal medium containing various supplements were used as culture media (A: XenoB110-gfp-luc2; B: Xeno284-gfp-luc2). Luciferase activities were measured at day-2. Data is the average of triplicate from three tumours and the standard deviation is represented by error bars. *p<0.05

FIG. 15: Examples of data showing use of the assay to test the viability/proliferation of gfp-luc2-labelled xenograft cells in a co-culture system. Xenograft cells were co-cultured with two cell types in (A) 2D culture model; and (B) 3D culture model. Luciferase activities of cells were measured at day-4. Data is the average of triplicate from three tumors and the standard deviation is represented by error bars. *p<0.05.

FIG. 16: Examples of data showing use of the assay for drug testing using PDXs cells, in accordance with an embodiment of the present invention. Experimental results from 2D cells, and 3D spheroids treated with a standard chemotherapy drug, cisplatin;

FIG. 17: Examples of data showing use of the assay to test the effects of single and combination therapy (radiotherapy and drug/inhibitor) on PDXs cells. (A) Graph showing percent viability of gfp-luc2-labelled xenograft cells after 72 hours treatment. (B) Heat maps of combination index (CI). Inhibitor, NVP-BGT226.

FIG. 18: Examples of data showing use of the assay to test ex vivo application in an inhibitor (NVP-BGT226) testing. (A) Relative viability calculated as normalized luminescence signal for gfp-luc2-labelled xenograft fragments, signal readings at 0, 24 and 48 hours time point. (B) Relative viability of gfp-luc2-labelled xenograft fragments measured at 0, 24 and 48 hours of inhibitor treatment.

FIG. 19: Examples of data showing in vivo monitoring of tumour progression over time. Data from bioluminescence imaging of gfp-luc2-labelled PDX model, (A) subcutaneous, (B) orthotopic and (C) metastatic;

FIG. 20: Examples of data showing application for drug testing using subcutaneous tumor model of gfp-luc2-labelled PDX. Experimental results showing in vivo tumor inhibition in response to treatment using standard chemotherapy drug, cisplatin; and

FIG. 21: Examples of data showing application for drug testing using metastatic tumor model of gfp-luc2-labelled PDX. Experimental results showing in vivo tumor inhibition in response to treatment using standard chemotherapy drug, cisplatin.

### DETAILED DESCRIPTION

The description of a number of specific and alternative embodiments is provided to understand the inventive features of the present invention. It shall be apparent to one skilled in the art, however that this invention may be practiced without such specific details.

It is to be understood that this invention is not limited to particular compositions, methods, and experimental conditions described, as such compositions, methods, and conditions may vary. It is also to be understood that the terminology used herein is for purposes of describing embodiments only and is not intended to be limiting.

Unless defined otherwise, all technical and scientific terms used herein have the same meaning as commonly understood by one of ordinary skill in the art to which this invention belongs. Thus for example, “xenografts” broadly refers to “a tissue graft or organ transplant from a donor of a different species to the recipient”; whereby PDX refers to patient-derived xenograft and NSG (NOD scid gamma) animal model denotes a non-human model with “severe combined immunodeficiency”.

Methods in Accordance with Embodiments of the Present Invention

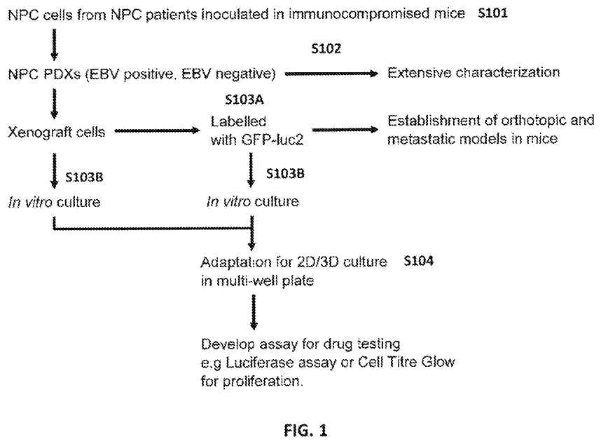

In a first aspect, the present invention discloses a method of preparing NPC-patient derived cells useful for further evaluation or studies. As shown in FIG. 1, NPC cells from NPC patients are inoculated in immunocompromised mice or a non-human animal model S101. Xenografts, referred herein as NPC PDXs are subjected to extensive characterisation at S102. Xenograft cells (after tissue dissociation and digestion process) can be subjected to labelling step S103A or directly to an in vitro culture process S103B. Cells that are subjected to labelling could be used to establish orthotopic and metastatic models in mice or suitable non-human models; or adapted for in vitro monolayer (2D), spheroid/organoid (3D) culture and/or ex-vivo xenograft tissue culture in multi-well plate format at S104. These cultures may be used for, but not limited to, developing assays for drug testing, and the like for further NPC studies.

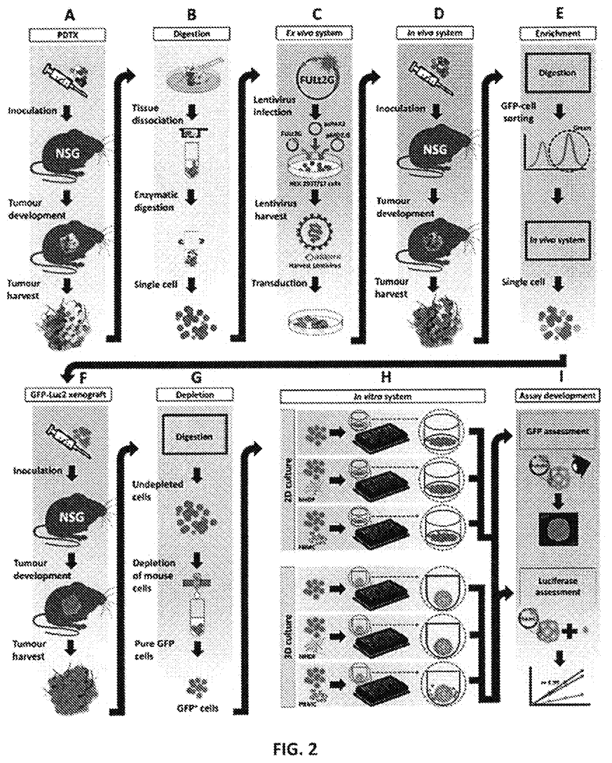

A detailed schematic diagram for the method shown in FIG. 1 in accordance with a preferred embodiment of the present invention is depicted in FIG. 2. As shown in FIG. 2, Stage A provides the steps involved in the development of NPC xenograft and tissue harvest. The second stage, Stage B depicts the steps involved in tissue dissociation and digestion to obtain cells of the xenograft. The cells are then subjected to a transduction process (ex-vivo system) at Stage C. The cells (gfp-luc2, upon labelled) are then inoculated and grown in a non-human model (in vivo system), more particularly in NSG mouse for tumour harvest at Stage D prior to selection at Stage E. The labelled cells are re-inoculated after the selection process for the tumour development and tissue harvest at Stage F. The next stage observes the depletion of mouse cells from cancer cell population (Stage 0) prior to subjecting the labelled cells for 2D and/or 3D co-culture studies. Cell proliferation and measurement analysis are then performed at Stage I for assay development.

In alternative embodiments, described herein are animals or models or grafted tissues (designated as “Xeno-”, each of which represents NPC patient derived PDXs, as discussed in the EXAMPLES and throughout description), and/or methods used to develop, validate/identify or optimize new or improved compounds, treatments, diets or therapies or the like; or to develop, validate or optimize new or improved test compounds, treatments, diets or therapies for the ability to prevent, reverse, ameliorate and/or inhibit NPC cancer growth in a bone or other metastatic-niche; developing or identifying agents against cancer cells, including candidate biomarkers, drug targets, chemical entities, validating and analysing the effects of drugs/bioactive compounds and candidate drugs.

In a further aspect, the present invention provides a method of evaluating the efficacy of an agent used to treat nasopharyngeal carcinoma (NPC) comprising the isolation of NPC cells from NPC patients; implanting/introducing said NPC cells in a non-human model, harvesting tumour xenografts and subjecting said xenografts to a tissue dissociation and digestion process to obtain single cells, and labelling said cells with fluorescent and/or luminescent protein i.e. gfp-luc prior to preparing/adapting the cells for use in further studies in carcinoma. In one embodiment, the isolated NPC cells may be characterised to identify its properties. In one embodiment, the cells may include proven Epstein-Barr virus (EBV) positive properties, and/or cells exhibiting EBV negative properties.

In another aspect, the present invention provides a luciferase-based assay to analyse the patient-derived xenografts (PDX) that expresses a fluorescent protein or a luciferase, or a combination thereof, for use in evaluating therapies comprising NPC cells.

In a further aspect, the present invention discloses, and with the support of experimental examples, a method of analysing the proliferation of xenograft cells, the method being advantageously sensitive and can specifically measure the real-time proliferation of xenograft cells both in vitro and in vivo. It is anticipated that the method of analysing the proliferation in accordance with the preferred embodiments of the present invention, can measure the xenograft cells growth enhancement resulted from the addition of growth supplements as well as from the effect of co-culturing with other human cell types, in addition to the ability to gauge the inhibition of cell viability induced by Cisplatin treatment. It is understood that the inhibition of cell viability may be carried out by other standard means of treatments or new agents to achieve the same purpose.

An analysis on the proliferation or viability of the xenograft cells obtained in accordance with a preferred embodiment of the present invention is shown under EXAMPLE 6, referred herein as “XenoLuc” or “XenoLuc Assay” or “XenoLuc Bioluminescence Cell Based Assay”. Results were obtained and the specificity of XenoLuc assay was assessed.

It is a further aspect of the present invention to provide a method for labelling cells without extended in vitro propagation; the method comprises: labelling of xenografts with gfp-luc2 marker using a lentiviral vector; and re-inoculation into non-human model i.e. mice after transduction to avoid cell death due to in vitro culture, to reduce risk of genetic alterations to the original cells during in vitro culture, to label xenograft cells which are difficult to culture in vitro and thereby to improve the success of labelling of the xenografts. In one embodiment, the lentiviral vectors include a combination of hexadimethrine bromide and inoculation with viral supernatant concentrate.

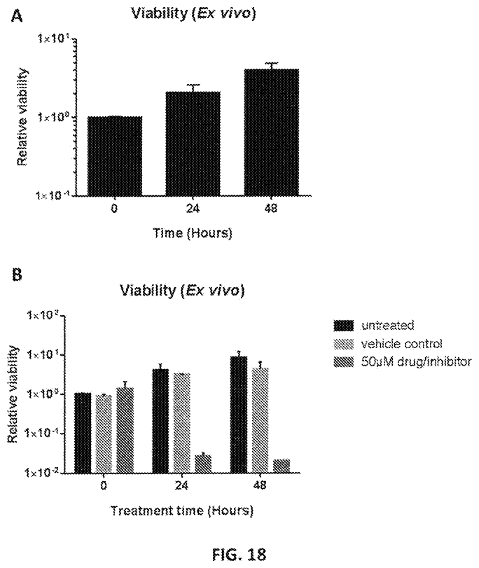

In another embodiment of the present invention, the method further includes culturing the NPC PDX cells in multi-well plates; adaptation of xenograft cells into short term in vitro culture in multi-well plates; whereby the growth conditions are optimised to allow the xenograft cells to grow in vitro in 2D, 3D conditions or ex-vivo into multi-well plates, more preferably in 96 and/or 384 well-plates. The optimization allows the cells to be used for multiple parallel assays in multi-well plate formats for, but not limited to, drug screening. Examples of in vitro 21) and 3D cultures and their applications are shown in FIG. 7B, FIG. 10 to 18.

The present invention further provides evaluating viability of NPC PDX cells grown in multi-well plates comprising: a) performing non-lytic luciferase assays of NPC PDX cells cultured in multi-well plates for drug screening for NPC; whereby the cancer cells alone or cancer cells co-cultured with other cells; and b) establishing non-lytic luciferase assay to measure cell viability in a non-destructive manner and in co-culture systems. The latter allows repeated measurement of cell viability over time without destroying the cells. It also enables measurement of cell viability of the tumour cells in the presence of other cells in co-culture systems and visualising as well as identifying the cancer cells that am GFP positive under fluorescent microscopy or confocal microscopy or other suitable techniques. Accordingly, it is anticipated that this measurement approach can be used in a method of cell counting to validate results of the luciferase assay.

In another embodiment, the present invention includes an assay comprising isolated NPC xenograft cells in vitro culture in multi-well plates co-cultured with fibroblasts, blood components or other cells. The isolated xenograft cells can be useful for evaluating/screening of inhibitors/drugs acting on the intracellular pathway; i.e. PI3K/Akt signalling pathway using NPC PDX that includes PIK3CA mutation, whereby it is evident from the experimental data and results examples that one of the xenografts, in particular (Xeno-284) harbours PIK3CA mutation and was adapted for drug testing. The evaluation and/or screening of drugs targeting EBV or EBV associated cancer or other EBV associated diseases can be performed by way of parallel testing of EBV negative NPC (Xeno-284) with EBV positive NPC (Xeno-B110).

### Xenograft Cells

In one embodiment, the present invention relates to PDXs cells/systems/models and/or derivatives, parental (unlabelled) and/or labelled, expressing a fluorescent protein, or a luciferase, or a combination thereof, for evaluating therapies comprising NPC (both EBV positive and EBV negative).

These patient-derived cells and xenografts in accordance with the embodiments of the present invention are useful as tools for determining the impact of agents on cellular biology, tumorigenesis, viability, apoptosis, and metabolic profiles; as well as for the discovery of new therapeutic targets and for the screening of novel molecular therapeutic agents.

Cells obtained from NPC PDXs referred herein as Xeno-284, Xeno-287, Xeno-B110, Xeno-G514, Xeno-GS17, Xeno-G518, and Xeno-G244 were established and characterised with methods and materials to be described under EXAMPLE 1-EXAMPLE 3 in this disclosure.

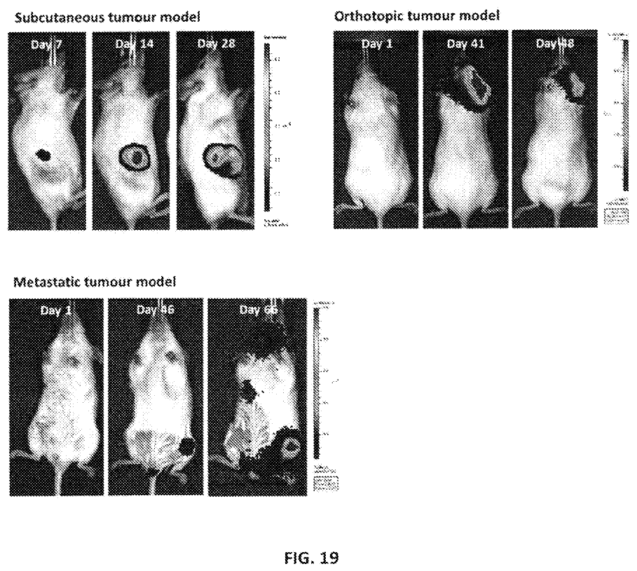

In another embodiment, the isolated xenograft cells can be used in monitoring tumour burden and/or metastasis for NPC model and evaluating candidate biomarkers/drug targets; evaluating drugs targeting metastatic NPC using PDX of NPC; and for evaluating drugs targeting invasive NPC using PDX of NPC-NPC gfp-luc2 cells inoculated into the mouse nasopharynx or into the left ventricle or other inoculation sites for metastatic models as shown in FIG. 19 to FIG. 21.

Suitably, it is within the scope of the present invention, to provide a method of evaluating the efficacy (such as elimination or killing or slowing the progress or spread of the primary cancer) of an agent, whereby said agent may be selected from one of the following: bioactive compound, chemical entity, biologics, candidate biomarkers, candidate drug targets, candidate drugs, or a combination thereof.

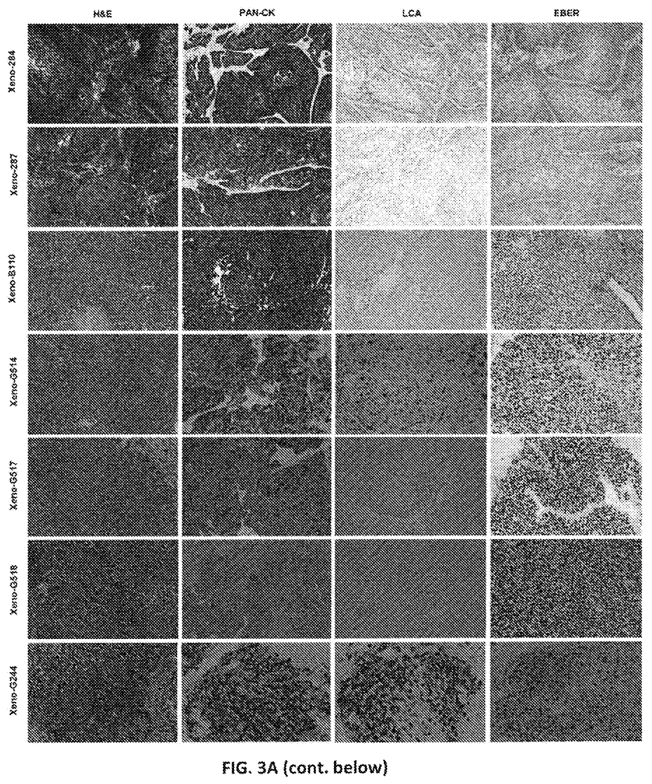

FIG. 3 to FIG. 7 provides the characterisation of the PDXs in accordance with the preferred embodiment of the present invention, as also described in EXAMPLE 1-3. It is found that at least 6 of the developed NPC PDXs were serially transplantable identified as Xeno-284, Xeno-287, Xeno-B110, Xeno-G514, Xeno-G517 and Xeno-G518, in which their characteristics are also shown in FIG. 3 and TABLE 1.

TABLE 1
Molecular characterization of PDXs.
(A) List of mutation of several genes identified in PDXs. (B) HLA-typing of PDXs.
(A) List of mutation identified in PDXs.

| Sample | NF-K [symbol] pathway | P [symbol] 3K/MAPK pathway | Mismatch repair pathway | Other pathway |
|---|---|---|---|---|
| Xeno-284 | | P [symbol] K3CA missense mutation | | HLA-A truncating mutation |
| | | (c.1633G > A, E [symbol] 45K) | | (c. [symbol] 37G > T, p.E113Te [symbol] ) |
| | | FGF [symbol] 2 missense mutation | | TP53 missense mutation |
| | | (c.1436G > C, p. [symbol] 479T), | | (c.209C > T, p. [symbol] 70V, |
| | | (c.14 [symbol] [symbol] G > C, p. [symbol] 471 [symbol] ) | | truncating mutation |
| | | | | (c. [symbol] 17delG, |
| Xeno-B110 | [symbol] YL [symbol] truncating mutation | P [symbol] EN inframe mutation | | p.V73W [symbol] Te [symbol] 50) |
| | (c. [symbol] 112C > A, p [symbol] 373Te [symbol] ) | (c.76 [symbol] _776del | | |
| | | AGAGTTCTTCCA, | | |
| | | p.E256_H2 [symbol] 9del) | | |
| Xeno-G517 | CYLD truncating mutation | | | |
| | (c.1461G > A, p.W [symbol] 87X) | | | |
| Xeno-G514 | NFKB [symbol] | | M [symbol] H [symbol] | |
| | (c. [symbol] 74C > T, [symbol] . [symbol] 192W) | | missense mutation | |
| | | | (c. [symbol] 121T > A, p. [symbol] 374 [symbol] ) | |

(B) HLA-genotyping (HLA-A and HLA-B) of PDXs.

| | HLA-A* | HLA-B* |
|---|---|---|
| Xeno-284 | 24: [symbol] 2, [symbol] 3:02 | 58:01, 13:02 |
| Xeno-B110 | 02:07 | 46:01 |
| Xeno-G514 | 11:01, 24: [symbol] 2 | 15:02, 3 [symbol] :05 |
| Xeno-G517 | 24:02, 24:07 | 15:02, 35:0 [symbol] |
| Xeno-G518 | 24:02, 24:07 | 15:02, 35:0 [symbol] |

[symbol]

indicates data missing or illegible when filed

FIG. 3 provides the experimental results based on the histological and IHC characterisation of PDXs.

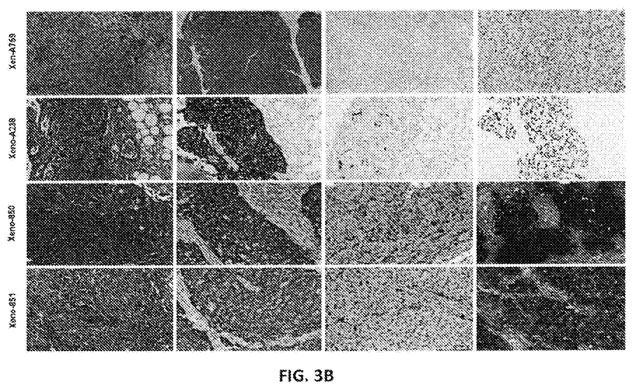

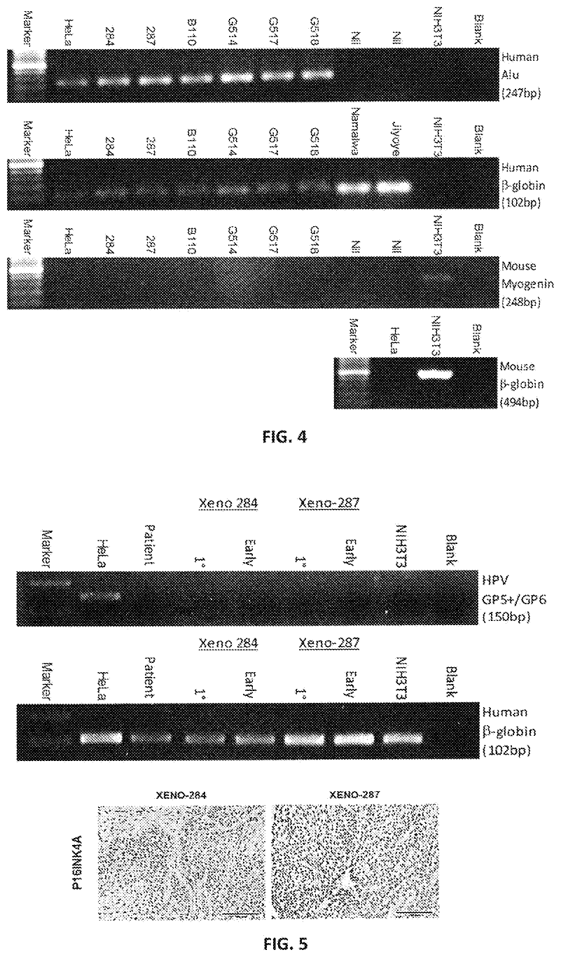

FIG. 4 provide examples of experimental results in relation to verifying the PDXs origin (human vs mouse) by means of PCR. In this experiment, HeLa, NIH3T3. Namalwa, Jiyoye and Blank are the control samples. In conjunction to this experiment, TABLE 2 below provides examples of DNA fingerprinting data for verification of genetic identity of PDXs with the corresponding patients.

TABLE 2
Examples of DNA fingerprinting data for verification of genetic identity of PDXs with the corresponding patients.

| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
|---|---|---|---|---|---|---|---|---|---|---|---|---|---|---|---|---|
| Sample | | | | | | | | | | | | | | | | |
| [symbol] | [symbol] | [symbol] , [symbol] | [symbol] , [symbol] | [symbol] | [symbol] , [symbol] | [symbol] , [symbol] | [symbol] | [symbol] , [symbol] | [symbol] | [symbol] , [symbol] | [symbol] , [symbol] | [symbol] | [symbol] | [symbol] | [symbol] , [symbol] | X, Y |
| [symbol] | [symbol] | [symbol] , [symbol] | [symbol] , [symbol] | [symbol] | [symbol] , [symbol] | [symbol] , [symbol] | [symbol] | [symbol] , [symbol] | [symbol] | [symbol] , [symbol] | [symbol] | [symbol] | [symbol] | [symbol] | [symbol] , [symbol] | [symbol] |
| [symbol] | [symbol] | [symbol] , [symbol] | [symbol] , [symbol] | [symbol] | [symbol] , [symbol] | [symbol] , [symbol] | [symbol] | [symbol] , [symbol] | [symbol] | [symbol] , [symbol] | [symbol] | [symbol] | [symbol] | [symbol] | [symbol] , [symbol] | [symbol] |
| [symbol] | [symbol] | [symbol] , [symbol] | [symbol] , [symbol] | [symbol] , [symbol] | [symbol] | [symbol] , [symbol] | [symbol] | [symbol] , [symbol] | [symbol] | [symbol] , [symbol] | [symbol] , [symbol] | [symbol] , [symbol] | [symbol] | [symbol] | [symbol] , [symbol] | X, [symbol] |
| [symbol] | [symbol] | [symbol] , [symbol] | [symbol] , [symbol] | [symbol] , [symbol] | [symbol] | [symbol] , [symbol] | [symbol] | [symbol] , [symbol] | [symbol] | [symbol] , [symbol] | [symbol] | [symbol] , [symbol] | [symbol] | [symbol] | [symbol] , [symbol] | X, [symbol] |
| [symbol] | [symbol] , [symbol] | [symbol] | [symbol] , [symbol] | [symbol] , [symbol] | [symbol] | [symbol] , [symbol] | [symbol] , [symbol] | [symbol] , [symbol] | [symbol] , [symbol] | [symbol] , [symbol] | [symbol] , [symbol] | [symbol] , [symbol] , [symbol] | [symbol] , [symbol] | [symbol] , [symbol] | [symbol] | X, Y |
| [symbol] | [symbol] , [symbol] , [symbol] | [symbol] | [symbol] , [symbol] | [symbol] , [symbol] | [symbol] | [symbol] , [symbol] | [symbol] , [symbol] | [symbol] , [symbol] | [symbol] , [symbol] | [symbol] , [symbol] | [symbol] | [symbol] , [symbol] , [symbol] | [symbol] , [symbol] | [symbol] | [symbol] | X, Y |
| [symbol] | [symbol] , [symbol] | [symbol] | [symbol] | [symbol] , [symbol] | [symbol] , [symbol] | [symbol] | [symbol] | [symbol] , [symbol] | [symbol] , [symbol] | [symbol] | [symbol] , [symbol] | [symbol] , [symbol] | [symbol] | [symbol] , [symbol] | [symbol] , [symbol] | [symbol] |
| [symbol] | [symbol] , [symbol] | [symbol] | [symbol] | [symbol] | [symbol] | [symbol] | [symbol] | [symbol] , [symbol] | [symbol] , [symbol] | [symbol] | [symbol] | [symbol] , [symbol] | [symbol] | [symbol] , [symbol] | [symbol] , [symbol] | X |
| [symbol] | [symbol] , [symbol] | [symbol] | [symbol] | [symbol] | [symbol] | [symbol] | [symbol] | [symbol] , [symbol] | [symbol] , [symbol] | [symbol] | [symbol] | [symbol] , [symbol] | [symbol] | [symbol] , [symbol] | [symbol] , [symbol] | X |

[symbol]

indicates data missing or illegible when filed

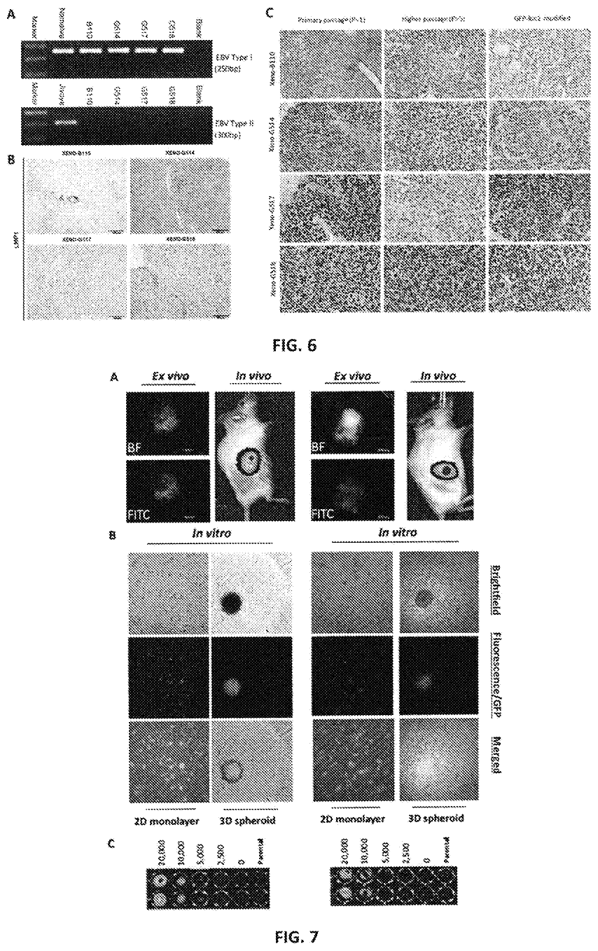

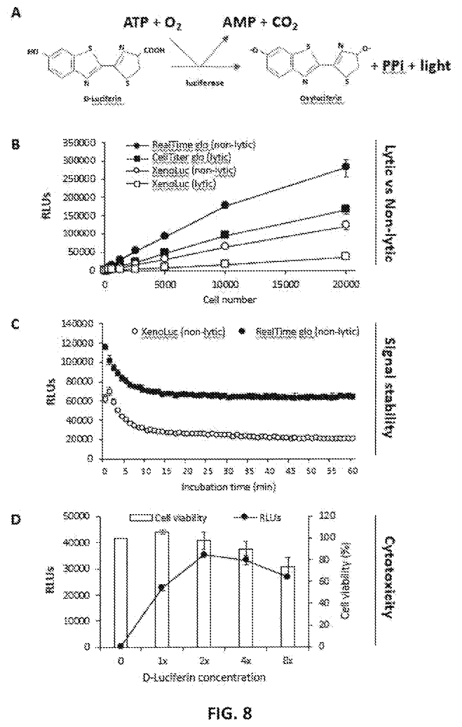

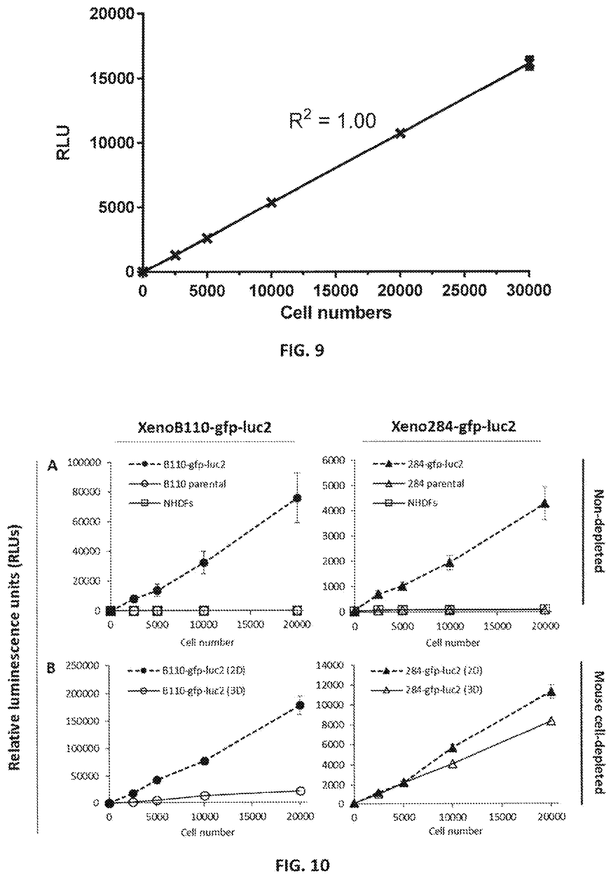

FIG. 5 provides the experimental results depicting characterisation of EBV negative PDXs, while FIG. 6 depicts the experimental results depicting further characterisation of EBV positive PDXs.

The molecular characterisation (mutations) and HLA of PDXs, in particular Xeno-284, Xeno-B110, Xeno-G517 and Xeno-G514 are shown in TABLE 1.

It is anticipated that the serially transplantable NPC PDXs developed in accordance with the present invention is not limited to the PDXs as designated in this disclosure, in which the scope of protection covers PDXs having characteristics similar to that of all PDXs described herein.

The following provides experimental examples in relation to the preparation of non-human model and the characterisation of the xenografts in accordance with an embodiment of the present invention. It should be noted that the experimental examples should not be construed as limitations to the scope of protection.

### Example 1

### Establishment and Characterization of NPC PDXs

### NPC Tumour Tissue Procurement, Processing and Xenograft Development

Tissue specimens were obtained from patients undergoing biopsy or surgery with informed consent obtained prior to the procedure. Specimen collection and usage was in accordance with the protocols approved by Medical Research Ethic Committee (MREC), Ministry of Health, Malaysia. Fresh tissue from the biopsy specimen was immediately placed in AQIX® RS (Aqix Limited) media to maintain tissue viability during transportation and were further processed for in vivo transplantation into NSG mice. The biopsy specimen was washed with cold phosphate buffered saline, cut into small fragments and implanted into mice subcutaneously (SC) or under the sub-renal capsule (RC). All mice were housed in specific pathogen free facility, maintained and used in accordance with the institutional guidelines and protocols which were approved by the Animal Care and Use Committee (ACUC), Ministry of Health, Malaysia. Tumour growth was closely monitored by manual palpation. Once positive tumour growth were detected, mice were sacrificed for tumour harvesting. Harvested tumours were divided into several portions for subsequent passaging in mice, characterizations and tissue repository. Subsequent generations of xenografts also underwent the same procedure as the first-generation xenografts. Serial passaging in vivo was maintained to ensure the continuity of the xenograft line.

### Histopathological and IHC Characterization

Serial sections (3-4 m thick) were prepared from formalin-fixed paraffin-embedded (FFPE) specimen and stained with hematoxylin and eosin (H&E) for histopathological evaluation. Tissue sections were also subjected to IHC using the following anti-human antibodies for the assessments of pan-cytokeratin (pan-CK, clone: AE1/AE3, DAKO) and leukocyte common antigen (LCA, clone: 2B11+PD726, DAKO). IHC staining was performed on the BondMax™ immunostainer (Leica Biosystems, Melbourne, Australia) by utilizing Bond Polymer Refine Detection (Leica Biosystems, Newcastle, United Kingdom). Marker expressions were visualized using diaminobenzidine (DAB) chromogen (brown) and hematoxylin (blue) was used as counterstain.

### Verification of Human Tissue Origin and Identity

The amplification of human Alu and mouse myogenin sequences were done to verify the human tissue origin of the PDXs. The human Alu sequence was amplified based on the method described by Steck et al and the mouse myogenin PCR followed the previous method by Marchiano et al. The primers used for human Alu were (forward) 5′-CGAGGCGGGTGGATCATGAGGT-3′ and (reverse) 5′ TTTTTGAGACGGAGTCTCG-3′, while the primers used for mouse myogenin were (forward) 5′-1TACGTCCATCGTGGACAGGA-3′ and (reverse) 5′-TGGGCTGGGTGTTAGCCTT′A-3′.PCR assays were carried out in separate reactions for each sequence using PCR GoTaqgGreen Master Mix (Promega, Madison, Wis., USA) which contained 1×PCR buffer, 1.5 mM MgCl2, 0.2 mM of dNTPs, 0.4 μM primers and IU GoTaq® a DNA polymerase.

DNA fingerprinting analysis using multiplex PCR of short tandem repeat (STR) elements was performed to determine the genotypes and to authenticate the resulting xenografts. STR profiling of 16 loci was performed using the AmpFISTR Identifiler PCR Amplification Kit (Applied Biosystem, Foster City, Calif., USA). PCR assay was performed on GeneAmp PCR System 9700 (Applied Biosystem, Foster City, Calif. USA). Cycling conditions for multiplex PCR: 1 cycle of 95° C. for 11 min, 28 cycles of 94° C. for 1 min, 59° C. for 1 min and 72° C. for 1 min, followed by 60° C. for 1 min and samples were hold at 4° C. until retrieved. Capillary electrophoresis was accomplished using ABI 3730 DNA Analyzer (Applied Biosystem, Foster City, Calif., USA). Analysis was performed using the GeneMapper® ID software (ver. 3.1. Applied Biosystem).

### Results

Referring to FIG. 3A and FIG. 3B, Transplantation of patients' tumour tissue into mice gave rise to 11 NPC PDX (Xeno-284, Xeno-287, Xeno-B110, Xeno-G514, Xeno-G517, Xeno-G518, Xeno-G244, Xeno-A759, Xeno-A238, Xeno-850 and Xeno-851). Out of these, 6 of the PDXs were serially transplantable (Xeno-284, Xeno-287, Xeno-B110. Xeno-GS14, Xeno-G517 and Xeno-G518). Histopathological characterization showed that these PDXs resemble the characteristics of the respective original patient's tumour. Further immunohistochemical phenotyping showed positive expression of the epithelial marker (pan-cytokeratin) which confirmed the epithelial origin of all PDXs. Now referring to FIG. 4, all PDXs showed positive amplification of human Alu sequences, which confirmed the human origin of the PDXs. DNA fingerprinting showed that the genotypes of each PDX is in concordance with their respective original patient's genotype—as shown in TABLE 2.

### Example 2

### Verification of EBV and HPV Status in NPC PDXs

### EBER ISH

EBER ISH was performed on FFPE sections for the detection of EBV infection. RNA probes directed against the EBERs transcripts were utilized for the detection of EBV. Additional probes were used as controls to assess RNA preservation and also background staining in tissue samples (RNA positive controls, RNA negative controls). All probes (Leica Biosystems, Newcastle, United Kingdom) were used in combination with Bond Polymer Refine Detection system. ISH staining was performed on the BondMax™ immunostainer using modified protocol (Norazlin et al., 2016). Marker expressions were visualized using DAB chromogen (brown) and, hematoxylin (blue) was used as counterstain.

### EBNA-2 EBV Typing

EBV typing was performed by nested PCR amplification of EBNA-2 regions as previously described by Hassan et al. The first PCR reaction was done to amplify a common region of EBNA-2. This was followed by two separate nested reactions which amplified distinctive regions of EBNA-2 using PCR product from the first reaction. Reaction mixture contained 1×PCR buffer, 1.5 mM MgCl2, 0.4 mM dNTPs, 0.4 μM primers and IU GoTaq® DNA polymerase (GoTaq® Flexi DNA Polymerase, Promega, Madison, Wis., USA).

### LMP1 IHC Staining

Latent membrane protein 1 (LMP1) staining was performed using anti-LMP1 antibody (clone: CS. 1-4). Staining was performed on the BondMax™ immunostainer (Leica Biosystems, Melbourne, Australia) by utilizing Bond Polymer Refine Detection or BOND Intense R Detection system (Leica Biosystems, Newcastle, United Kingdom).

### PCR for the Detection of HPV GP5+/GP6

PCR analysis to detect the presence of HPV was carried out by using HPV consensus primers GP5+/GP6+ as previously described by Antonsson et al., 2010 and Husman et al., 1995. All PCR assays were carried out in a final volume of 25 μl reaction mixture which contained 150 ng sample DNA. Appropriate positive and negative controls were included for every PCR assay. Reactions containing no template DNA served as template blank (TB) negative controls. DNA was amplified in reaction mixture which contained 1×PCR buffer, 2 mM MgCl2, 0.2 mM deoxynucleotide (dNTP) solution mix of dATP, dCTP, dGTP and dUTP (dTTP was substituted with dUTP to control carry-over of PCR product), 0.5 μM primers and 1 U GoTaq® Flexi DNA Polymerase.

### IHC of P16INK4A

IHC staining of p16INK4A marker was performed using anti-p16INK4a antibody (clone: 2D9A12, Abcam). Staining was performed on the BondMax™ immunostainer (Leica Biosystems, Melbourne, Australia) by utilizing Bond Polymer Refine Detection or BOND Intense R Detection system (Leica Biosystems, Newcastle, United Kingdom).

### Results

EBER in situ hybridization staining for detection of EBV showed that Xeno-B110, Xeno-G514, Xeno-G517, Xeno-G518 and Xeno-G244 were positive for EBV. However, Xeno-284 and Xeno-287 were negative for EBV as shown in FIG. 3A.

Further, now referring to FIG. 6, all EBV positive PDXs showed positive amplification for EBV Type I. IHC staining of LMP1 showed high expression in Xeno-G514. EBV were maintained throughout serial passages in the PDXs. In addition, EBV status were also maintained in gfp-luc2-modified PDXs.

EBV negative PDXs (Xeno-284 and Xeno-287) were further tested for presence of HPV via PCR and IHC staining of P16INK4A. As shown in FIG. 5, test results indicate that HPV was not detected in both PDXs. There is no amplification of HPV GP5+/GP6+ sequences as well as no over-expression of the P16INK4A marker.

### Example 3

### Molecular Characterization of NPC PDXs

### Sanger Sequencing

Mutation status of selected genes were verified by aligning sequencing reads of PDXs samples to human reference genome (hg19). Sanger sequencing was performed using primers designed using Primer3 software (http://www.bioinformatics.nl/primer3plus). PCR amplification was conducted using GoTaq Green Master Mix (Promega). Sequencing was performed with ABI BigDye Terminator v3.1 (Life Technologies). The sequence chromatograms were viewed using Chromas ver.2.5.1 software (Technelysium Pty Ltd).

### HLA-Typing

High resolution HLA genotyping for Class I (HLA-A and HLA-B) was carried out using LabType XR kit (One Lambda). Briefly, 20 ng of DNA was used as starting material for the amplification of exons 2, 3, 4 and 5 of both HLA-A and HILA-B. Amplified DNA were then subjected to denaturation, followed by hybridization to sequence specific probes and lastly, labeling with phycoerythrin conjugated streptavidin (SAPE). Data acquisition was performed with LABScan3D and analysis was done with HLA Fusion (ver 4.1).

### Results

Sanger sequencing was performed on selected genes reported to be associated with NPC. Our results, as shown in TABLE 1(A), identified mutations in PDXs which are related to the NF-KB. PI3K/MAPK, mismatch repair pathway or other pathway such as p53. HLA-genotyping showed presence of HLA-alleles (A*02:07, B*46:01, 3*58:01) which are known risk alleles in NPC—TABLE 1B.

### Example 4

### Establishment of GFP-luc2 PDXs

### Plasmid Construct and Reagents

Lentiviral constructs and gfp-luc2 DNA transfer plasmid (FuL2tG) were used. RPMI 1640 (#31800-022), fetal bovine serum (FBS) (#10082-139), GlutaMAX supplement (#35050-061). 0.05% Trypsin-EDTA (#25300-120), Pen-Strep (#15140-122), Pen-Strep-Fungizone (#15240-062), B-27 supplement (#17504-001), Insulin-Transferrin-Selenium (ITS) (#41400-045), Fibroblast growth factor (FGF)-Basic (bFGF) (#PHG0261), and Epidermal growth factor (EGF) (#PHG0311) were obtained from Gibco, USA. Hydrocortisone (#H0135), Collagenase Type II (#C6885), DPBS (#D5652), HEPES (#H3375), and Sodium Bicarbonate (#55761) were purchased from Sigma-Aldrich, USA. DNase 1 (#90083) and Lipofectamine 3000 (#L3000015) were purchased from Thermo Fisher Scientific, USA. Collagenase/Dispase (#11097113001) was obtained from Roche, USA. Rho kinase (ROCK) inhibitor (#SCM075) and Polybrene (#TR-1003-G) were obtained from Merck Millipore. USA. XenoLight D-Luciferin substrate (#122799) was purchased from PerkinElmer, USA and stored in small aliquots at −20° C. in the dark. CellTiter 96 AQueous One Solution Cell Proliferation Assay (MTS) (#G3580), CellTiter-Glo Luminescent Cell Viability Assay (#G7571), and RealTime-Go MT Cell Viability Assay (#G9711) were from Promega, USA. Cisplatin (#15663-27-1) was purchased from Acros Organics, USA. RBC lysis solution was purchased from Qiagen, USA. All reagents were dissolved, stored, and used according to the manufacturer's instruction.

### Lentiviral Production and Cells Transduction

1×10⁶ HEK 293T cells were seeded on 10 cm culture dish and incubated for 24 hr. The lentiviral transfer vector FuL2tG together with packaging and envelope plasmids as mentioned above were combined at a ratio of 4:2:1:1, respectively and mixed with Lipofectamine 2000 for transfection according to the manufacturer's protocol. The viral supernatant was collected at 24-72 hr post-transfection and cleared by centrifugation at 1500 rpm for 5 min at 4° C. followed by a filtration using PVDF MillexHV filter. 0.45 μm (Millipore #SLHV033RS). The filtered lentivirus supernatant was then concentrated using Lenti-X Concentrator (Clontech #PT4421-2) according to the manufacturer's instruction. For the cell transduction, the xenograft cells were seeded on a 10 cm culture dish and transduced with the concentrated lentivirus at MOI 2.0 in the presence of 10 μg/mL Polybrene for 24 hr.

### Establishment of gfp-luc2 PDXs

The gfp-positive transduced cells were harvested and sorted using flow cytometry (BD FACS Aria III). The cells were stained with H2kd-PE anti-mouse antibody (BD Pharmigen, Clone SF1-1.1) to exclude the mouse cells. The human cell population enriched gfp-positive xenograft cells were inoculated into NSG mice, which then form the modified xenografts. Fluorescence images were captured either using Nikon AZM100 stereomicroscope (Nikon, Japan) or IN Cell Analyzer 2000 (GE Healthcare, USA) while the luminescence images were captured using IVIS in vivo imaging system (PerkinElmer).

### Results

FIG. 7 shows an example of imaging results obtained using an assay method in accordance with an embodiment of the present invention; which will be described herein. Our results showed that we have successfully established gfp-luc2 PDXs using the aforementioned assay method. The PDXs tumours/cells express gfp-protein as shown by data (A&B) acquired using the fluorescence microscopes (FITC images). Our data also shows that the PDXs tumours/cells express luc2-protein based on the luminescence signals which were detected using the IVIS imaging system (A&C).

### Example 5

### Optimization and Characterization of In Vitro Assays for 2D and/or 3D

### Cell Culture Conditions for 2D, 3D and Ex-Vivo

Tumour tissue from PDXs were processed as previously described by our group (Hoe et al., 2017). The tumours were minced and incubated with the appropriate dissociation solution. After that, RPMI 1640 basal medium (containing 5-10% heat-deactivated Fetal Calf Serum (Gibco® Life Technologies, USA), 1× Glutamax (Gibcol® Life Technologies, USA), 1× of Antibiotic/Antimycotic (Gibco® Life Technologies, USA), 1×B-27 Supplement (Gibco® Thermofisher Scientific, USA), 1× Insulin Transferrin-Selenium-A (Gibco® Thermofisher Scientific, USA), 0.5 mg/ml Hydrocortisone (Gibco®: Life Technologies, USA), 5-10 μM ROCK Inhibitor (Millipore, USA), 5-10 ng/ml Epidermal Growth Factor (Gibco® Life Technologies, USA) and 5-10 ng/ml Basic Fibroblast Growth Factor (Gibco®, Life Technologies, USA)) were added and cells were filtered through 40 μm nylon mesh cell strainer (BD Falcon, USA). The cell suspension was centrifuged at 800 rpm for 5 min. RBC lysis solution was added and centrifuged at 800 rpm for 5 min. The cells were then processed with the mouse cell depletion kit (MACS Miltenyi Biotec #130-104-694) following the manufacturer's instruction to remove mouse cells contamination. Cells were cultured in the above mentioned growth medium for 2D monolayer culture and 3D spheroid culture (with or without supplements). The cells were trypsinized using 0.05% Trypsin-EDTA at 70-80% confluence and were sub-cultured at 1:3 dilution.

For ex-vivo culture, PDXs tumour tissue was cut into small cubic fragment with the diameter of ˜3 mm. Subsequently, solid tissue fragments were placed into 96-well white clear bottom plate with 100 uL of RPMI 1640 complete medium and placed in 37° C. incubator with 5% CO₂ for 2 hours prior to further testing.

For adaptation of PDXs cell into multi-well plate formats, cell seeding density was adjusted according to the size of the respective multi-well plate.

### Adaptation into 2D, 3D or Ex-Vivo Cultures

For adaptation of PDXs cell into serially passageable 2D culture, derivatives of PDXs (single cells, spheroid and tissue fragment explant) were grown in RPMI 1640 complete media.

It is anticipated that the supplements concentration may vary, preferably within the ranges as follows: 0-25% heat deactivated calf serum, 0-5× Glutamax. 0-5× of Antibiotic/Antimycotic, 0-5×B-27 Supplement, 0-5× Insulin Transferrin-Selenium-A, 0-5 mg/mil Hydrocortisone, 0-50 μM ROCK Inhibitor, 0-50 ng/ml Epidermal Growth Factor and 0-50 ng/ml Basic Fibroblast Growth Factor.

### Characterization of PDXs Derivatives in In Vitro Culture

PDXs cells (2D and 3D) were characterized to ensure that the properties of the original PDXs were maintained (H&E, CK, EBER).

### Results

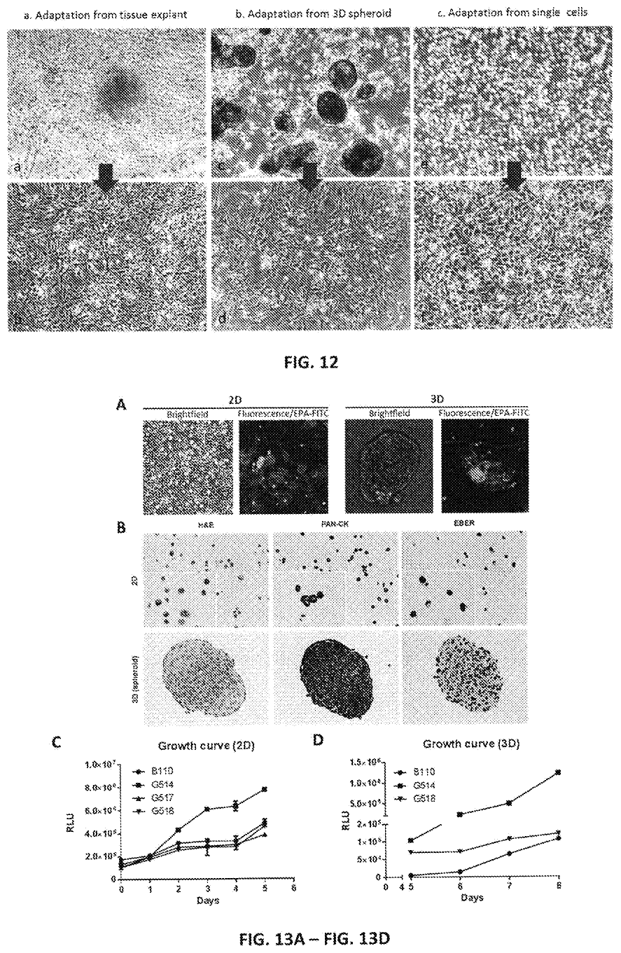

The above-mentioned conditions used were successful in establishing and maintaining short term culture and serially passaging of PDXs cell derivatives in vitro (FIG. 12 to FIG. 13). With reference to FIG. 13A and FIG. 131B, the characterization of the PDXs cell derivatives showed positive expression of epithelial marker (EPA, Pan-CK) and EBER. Short term culture viability analysis of PDXs cell derivatives showed that cells can grow and proliferate in vitro using the above-mentioned culture conditions as shown in FIG. 13C and FIG. 13D.

### Example 6

### Establishment and Validation of XenoLuc Assay

### XenoLuc Assay

XenoLuc assay, which is our in-house non-lytic luciferase assay was performed as follows for both 2D and 3D culture experiments. At each time point. 2×D-Luciferin substrate diluted in RPMI-10 was dispensed into each well containing cell culture at 1:1 ratio. The plate was gently agitated and incubated at room temperature in the dark for 10 min to stabilize the luminescence. The luminescent signal of each plate was then read by EnVision multi-label plate reader (PerkinEimer) using the ultrasensitive mode. Following the completion of a time-point reading, the D-Luciferin solution was removed from the same well. The cells were washed two times gently with 200 μL RPMI-10, and then replenished with 100 μL of fresh complete media until the subsequent reading. Alternatively, the images of gfp-expressing xenografts from each well were captured using IN Cell Analyzer 2000, and the GFP fluorescence intensity was measured and compared using the IN Cell Investigator software. For the lytic-based format of XenoLuc assay, the protocol described by Oba and co-workers (2003) was modified and adapted. Briefly, PBS supplemented with 1% Triton X-100 and 1× protease inhibitor cocktail (Millipore #539134) was used as cell lysis buffer and the lysis was performed for 10 min. After lysis, the assay buffer made up of 5 mM MgCl2 and 100 mM Tris-HCl (pH7.8) containing 2× DLuciferin substrate was added to the well to generate the luminescence. The plate was immediately measured after 5-min incubation at room temperature in the dark.

### Method for Viability/Toxicity/Proliferation Assays (MTS, RealTime™ Glo, CellTiter-Glo®)

Other assays for evaluating cell viability/toxicity/proliferation using CellTiter 96® One Solution (MTS), RealTimer™ Glo and CellTiter-Glo® were done using manufacturer's protocol.

### Results

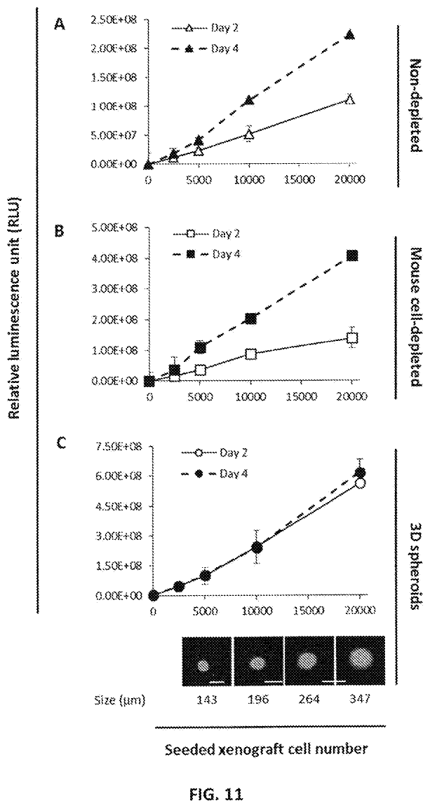

The specificity of XenoLuc assay was assessed by checking if this assay detects the luminescent signal only from luciferase-expressing cells. With reference to FIG. 10, luminescence was detected in luc2-modified xenograft cells but not in parental xenograft cells and NHDFs, indicating the high specificity of XenoLuc assay. As compared to the non-depleted xenograft cells, the mouse-depleted xenograft cells exhibited higher luminescent signal at the same number of tested cells. Since this assay involves the enrichment of viable luc2-bearing human xenograft cells, this further highlights the specificity of the assay design towards detecting signals from human cells, but not the mouse cells. Verification via flow cytometry also indicated that almost all mouse cells were removed from the xenografts using the mouse cell depletion kit (data not shown). To examine the assay sensitivity, two-fold serial dilutions of cells were plated and the readings were taken after 4 days. FIG. 11 shows the number of cells plated has a positive linear correlation with the luminescence both in 2D and 3D culture models of which the lowest seeding density tested was 2,500 cells/well in a 96-well plate.

In one embodiment, the XenoLuc assay in accordance with the preferred methods of the present invention provides specific, sensitive, rapid, and cost-effective for measuring the growth of luciferase expressing cells in a co- or multiple-culture system. The assay is suitable to be used in the tumour micro-environmental studies and drug screening in the complex 3D co-culture models. With this assay, the growth of NPC cells described in this document can be observed in both 2D and 3D models.

### Example 7

### Applications of PDXs Derivatives (Cell Viability/Toxicity/Sensitivity/Proliferation)—In Vitro

We evaluated the suitability of the assay for inhibitor/drug screening and co-culture study in both 2D and 3D culture models. The xenografts were first harvested and prepared into 2D or 3D or ex vivo, and treated with various concentrations of drugs or inhibitors/growth supplements or different radiation doses for 72 hr and followed by the XenoLuc assay. For 2D culture format, 100 μL of xenograft cell suspension in complete medium containing 10,000 cells was seeded overnight into each well of ViewPlate-96 Black plate (PerkinElmer, #6005182). Similar amount and seeding number of xenograft cells were plated in 3D culture conditions using Spheroid microplate-96 black plate (Corning #CLS4520). For ex vivo system, tumour was cut into small cubic fragment with the diameter of ˜3 mm and seeded into 96-well white clear bottom plate with 100 uL of complete medium.

For co-culture system, xenograft cells were seeded in multi-well plate format with normal human dermal fibroblast (NHDF) or peripheral blood mononuclear cells (PBMCs). Prior to co-culturing, NHDF or PBMCs were gamma-irradiated at 35 Gy. For both 2D and 3D cultures, 10,000 xenograft cells were seeded simultaneously together with the irradiated co-culture partner cells at 1:1 ratio into the same well. After incubation for desired periods of time, XenoLuc assay was performed to measure specifically the growth and proliferation of xenograft cells in the presence of either irradiated NHDF or PBMCs.

The method and embodiments of the present invention therefore provide a stable and serially transplantable continuous NPC cells produced from xenografts which can be grown and maintained in non-human model for both short term or long-term period. The cell line and the tumours that the xenografts produce may be used as model systems for study mechanisms; for instance, but not limiting to: metastatic behaviour and for testing, and screening for effective new anti-cancer drug/bioactive compound therapies. Understandably, further analysis or studies using the cell line or xenograft in accordance to the present invention may be accomplished by, for instance, introducing into a non-human animal model having the cell line a type of drug in an effective amount and analysing said animal to determine the effects of said drug against the tumour.

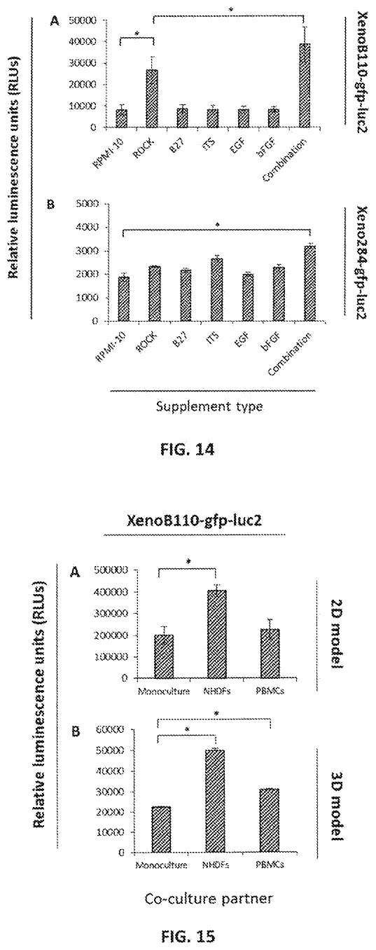

Experimental examples in relation to in vitro applications using the PDXs prepared in accordance with the embodiments of the present invention, more particularly for drug testing are shown in FIG. 14 to FIG. 18.

### Results

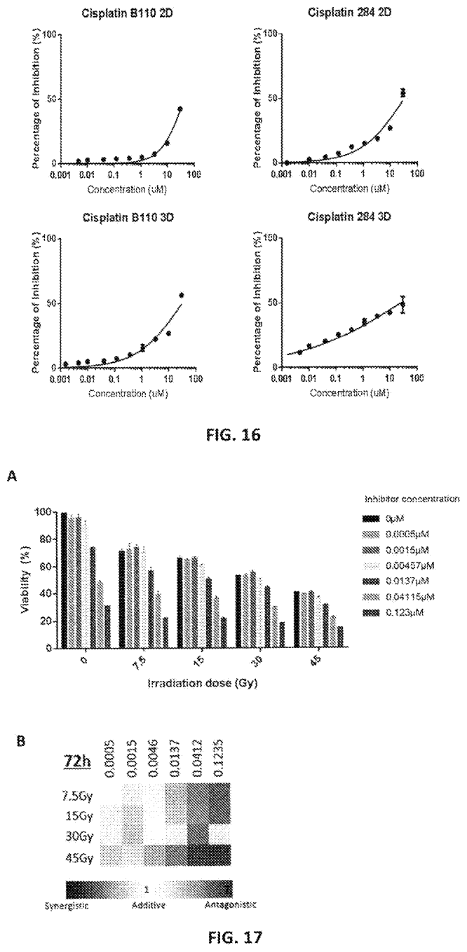

Our data support the use of the PDXs derivatives in both 2D and 3D culture models for assessing the effect of an agent on cell viability/toxicity/sensitivity/proliferation. With reference to FIG. 14 to FIG. 18, the effects of single or combination agent or co-culture on cell viability/toxicity/sensitivity/proliferation could be assessed using the aforementioned in vitro models. Cell viability was significantly enhanced by using combination of supplements or co-culture with other cells as depicted by the measured luminescence signals (FIG. 14 & FIG. 15). Treatment of PDXs derivatives using chemo-drug/inhibitor/radiotherapy were shown to induce inhibition on proliferation or reduce viability which can be used as a measure of sensitivity to said agent or toxicity of said agent (FIG. 16 to FIG. 18).

### Example 8

### Establishment of In Vivo PDXs Model (Subcutaneous, Orthotopic and Metastatic) and their Applications for Drug Testing

All experiments were performed in accordance with national regulations and were approved by the AC UC of Ministry of Health, Malaysia. NSG mice, 6-8 weeks old were anaesthetized using Ketamine-Xylazine (100 mg/ml and 10 mg/ml).

For orthotopic model, 2×10⁶ cells of gfp-luc2 cells were resuspended in 30 μl of RPMI and mixed with matrigel at 1:1 ratio and injected into the nasopharynx (Smith et al. 2011).

To test the effect of drug on tumour growth inhibition, subcutaneous and/or metastatic model were used. For subcutaneous model, 2×10⁵ cells of gfp-luc2 cells were resuspended in RPMI and mixed with matrigel at 1:1 ratio and injected into the right flank. For metastatic model, 2×10⁵ cells of gfp-luc2 cells were resuspended in 100 μl of RPMI and injected into left ventricle. Weekly bioluminescence imaging (BLI) were carried out to monitor the progress of tumour growth using VIS Spectrum.

When tumour volume reached to 50-150 mm³ or once the signal intensity reached 1×10⁴ (average radiance, p/sec/cm²/sr) mice were randomised into control or treatment group. Treatment commenced 35 days post inoculation. Mice were treated with vehicle (0.9% NaCl) or cisplatin (2 mg/kg) via intraperitoneal (i.p.) injection, weekly for 3 weeks. Tumour volume was measured thrice weekly using callipers and BLI was carried out weekly. Mice were observed daily for general wellbeing and tumour burden.

### Results

To enable in vivo tracking of tumour growth and metastasis, the PDX cells were transduced with a reporter gene expressing green fluorescent protein (GFP) and luciferase as mentioned previously. The progression of the tumours over time following subcutaneous, orthotopic or intra-cardiac injection could be imaged and monitored using in vivo bioluminescence imaging as shown in FIG. 19.

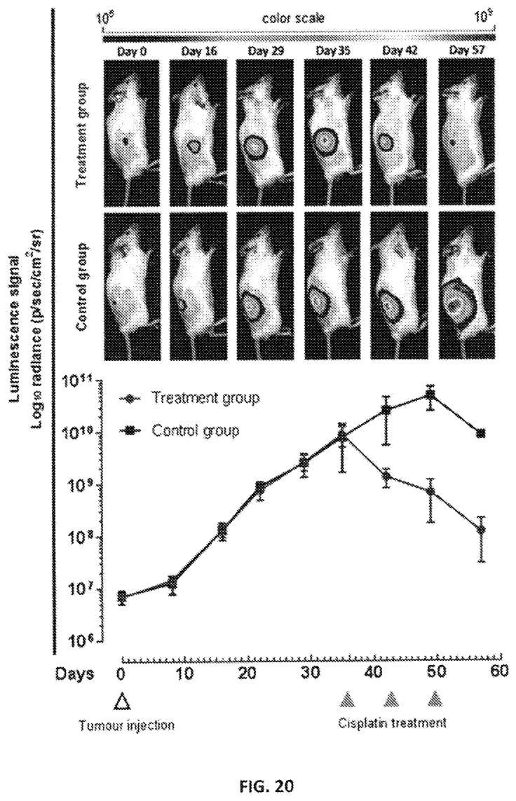

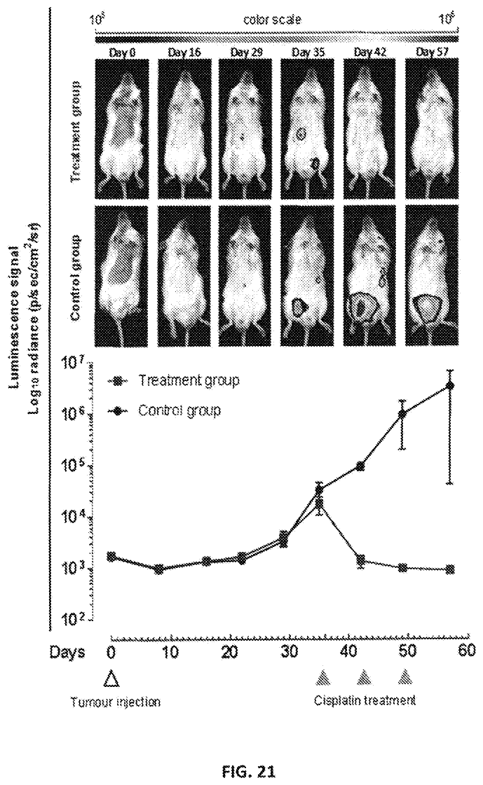

Subcutaneous injection of gfp-luc2-labelled PDX cells resulted in localized tumour growth at the injection area. Intra-cardiac injection of the gfp-luc2 PDX cells resulted in metastatic tumour deposits. With reference to FIG. 20 and FIG. 21, cisplatin treatment of both subcutaneous and metastatic model resulted in tumour regression as measured by the luminescence signals intensity.

It is anticipated that the methods described herein, although preferably adapted for NPC cells, it would be obvious for a person skilled in the art that the same methods may be applied for obtaining and analysing cancer cells other than that of NPC.

From the foregoing, it would be appreciated that the present invention may be modified in light of the above teachings. It is therefore understood that, within the scope of the appended claims, the invention may be practiced otherwise than as specifically described.

1. A method of utilising nasopharyngeal cancer (NPC) cells to develop assays to evaluate the efficacy of an agent against NPC comprising the steps of:
inoculating patient-derived NPC cells in an immunocompromised non-human model;
harvesting NPC xenograft tumour from the non-human model;
subjecting the NPC xenograft tumour to tissue dissociation and digestion process to obtain single NPC xenograft cells;
labelling the single NPC xenograft cells with gfp-luc2 marker using a lentiviral vector system in a transduction process;
inoculating the gfp-luc2 labelled NPC xenograft cells into a non-human model prior to selection of transduced cells;
harvesting gfp-luc2 labelled NPC xenograft tumour from the non-human model for selection of transduced cells;
re-inoculating the selected gfp-luc2 labelled NPC cells into a non-human model;
harvesting gfp-luc2 labelled NPC xenograft tumour from the non-human model and depleting any non-human cells to obtain gfp-luc2 labelled NPC xenograft cells;
growing the gfp-luc2 labelled NPC xenograft cells in in vitro culture and optimizing said culture into multi-well plates for use in assays to evaluate the efficacy of an agent against nasopharyngeal cancer.

2. The method as claimed in claim 1, wherein the lentiviral vector system includes the combination of hexadimethrine bromide and spinoculation with viral supernatant concentrate for the transduction process.

3. The method as claimed in claim 1, wherein the step of inoculating the gfp-luc2 labelled NPC xenograft cells into a non-human model prior to selection of transduced cells is performed to avoid the need for prolonged in vitro culture and to reduce risk of genetic alterations to the cells during in vitro culture.

4. The method as claimed in claim 1, wherein the method further comprises the step of evaluating the viability of the gfp-luc2 labelled NPC xenograft cells grown in multi-well plates; said evaluation comprising performing non-lytic luciferase assays of the gfp-luc2 labelled NPC xenograft cells cultured in multi-well plates for drug screening for NPC; establishment of a non-lytic luciferase assay to measure cell viability in a non-destructive manner which could be used in mono-culture or in co-culture systems.

5. The method as claimed in claim 1, wherein the NPC xenograft tumour harvested from the non-human model is characterized to determine whether the NPC cells provide a sufficient representation or duplication in the characteristics of tumours for use in further cancer studies.

6. The method as claimed in claim 1, wherein the step of inoculating the gfp-luc2 labelled NPC xenograft cells into a non-human model prior to selection of transduced cells produces an in vivo model utilised to monitor tumour burden and/or metastasis and to evaluate at least one of the following: candidate biomarkers, candidate drug targets, effects of drugs/bioactive compounds, a new chemical entity, candidate drugs, irradiation and/or other therapeutic agents, and studies on gene function, or a combination thereof.

7. The method as claimed in claim 1, wherein the NPC cells include EBV positive cells.

8. The method as claimed in claim 1, wherein growing the gfp-luc2 labelled NPC xenograft cells in in vitro culture and optimizing said culture into multi-well plates includes into 96-well plates or 384-well plates.

9. A method of evaluating the efficacy of an agent against nasopharyngeal cancer (NPC) comprising the steps of:
preparing a non-human model; whereby the non-human model carries cells obtained from a patient-derived NPC xenograft;
labelling the cells with gfp-luc2 marker using a lentiviral vector system in a transduction, process; and
growing the cells in in vitro 2D and 3D cultures and ex-vivo culture; including optimizing the cultures into multi-well plates for use in screening and/or evaluation assays, in monoculture or co-culture systems.

10. The method as claimed in claim 9, wherein the in vitro culture comprises: 0-25% heat deactivated calf serum, 0-5× Glutamax, 0-5× of Antibiotic/Antimycotic, 0-5×B-27 Supplement, 0-5× Insulin Transferrin-Selenium-A, 0-5 mg/ml Hydrocortisone, 0-50 μM ROCK Inhibitor, 0-50 ng/ml Epidermal Growth Factor and 0-50 ng/ml Basic Fibroblast Growth Factor.

11. The method as claimed in claim 9, wherein the in vitro culture comprises: 5-10% heat deactivated calf serum, 1× Glutamax, 1× of Antibiotic/Antimycotic, 1×B-27 Supplement, 1× Insulin Transferrin-Selenium-A, 0.5 ng/ml Hydrocortisone, 5-10 μM ROCK Inhibitor, 5-10 ng/ml Epidermal Growth Factor and 5-10 ng/ml Basic Fibroblast Growth Factor.

12. The method as claimed in claim 9, wherein the agent is selected from one of the following: drug, bioactive compound, chemical entity, biological agent, irradiation or a combination thereof.

13. The method as claimed in claim 9, wherein evaluating the efficacy of an agent against NPC includes studying the effects of the agent in eliminating, killing, and/or slowing the growth of cancer cells.

14. The method as claimed in claim 9, wherein at least a cell is EBV-positive.

15. A non-human in vitro model and in vivo model adapted for evaluating the efficacy of an agent against nasopharyngeal cancer (NPC) comprising at least one serially transplantable patient-derived NPC xenograft; Xeno-284, Xeno-287, Xeno-B110, Xeno-G514, Xeno-G517, Xeno-G518, Xeno-G244; said xenograft express at least one type of fluorescent protein and/or a luciferase wherein the xenograft is labelled with a gfp-luc2 marker using a lentiviral vector system in a transduction process, inoculated into a non-human model for tumour development prior to tumour harvesting for selection of transduced cells to be re-inoculated into a non-human model and harvesting cells established from the selected re-inoculated cells for further propagation, analysis or for adaptation into 2D and 3D culture.

16. The non-human in vitro model and in vivo model of claim 15, wherein the serially transplantable patient-derived NPC xenograft; Xeno-284, Xeno-287, Xeno-B110, Xeno-G514, Xeno-G517, Xeno-G518, Xeno-G244; exhibits mutations in nuclear factor-kappa-light-chain enhancer of activated B-cells (NF-κB), phosphatidylinositol 3′-kinase (PI3K) and/or, mitogen-activated protein kinase (MAPK) and/or mismatch repair (MMR) pathways and/or other pathways (i.e. p53) and/or expression of EBV LMP-1.

17. The non-human in vitro model and in vivo model of claim 15, wherein the serially transplantable patient-derived NPC xenograft; Xeno-284, Xeno-287, Xeno-B110, Xeno-G514, Xeno-G517, Xeno-G518, Xeno-G244; exhibits either one of the following genomic characteristics: CYLD (c.1112C>A, p.Ser371Ter); CYLD (c.1461G>A, p.W487X); NFKB1 (c.574C>T, p.Arg192Trp); PTEN (c.765_776 delAGAGTTCTTCCA, p.Glu256_His259del); PIK3CA (c.1633G>A, Glu545Lys); FGFR2 (c.1436G>C, p.Arg479Thr); FGFR2 (c.1411G>C, p.Glu471Gln); TP53 (c.217delG, p.Val73TrpfsTer50); MLH1 (c.1121T>A, p.Leu374Gln); HLA-A (c.3376>T, p.Glu113Ter); HLA-A*24:02, 03:02, HLA-B*58:01, 13:02; HLA-A*02:07, HLA-B*46:01; HLA-A*11:01, 24:02 HLA-B*15:02, 35:05; HLA-A*24:02, 24:07 HLA-B*15:02, 35:05.

18. The non-human in vitro model and in vivo model of claim 15, wherein at least one of the serially transplantable patient-derived NPC xenograft; Xeno-284, Xeno-287, Xeno-B110, Xeno-G514, Xeno-G517, Xeno-G518, Xeno-G244; tested EBV positive.

19. The non-human in vitro model and in viva model of claim 15, wherein the in vivo model can be used in determining tumour progression over time and/or identifying resistance and/or sensitivities of the cancer cells against candidate biomarkers, candidate drug targets, effects of drugs/bioactive compounds, a new chemical entity, candidate drugs, biological agent, irradiation and/or other therapeutic agents, and studies on gene function.

20. The non-human in vitro model, and in vivo model of claim 15, wherein in in vitro assays, the xenograft cells are co-cultured with immune or other stromal cells.

21. A method for labelling nasopharyngeal cancer (NPC) cells that are difficult to culture in vitro and without extended in vitro propagation, the method comprises the steps of;
labelling NPC xenografts with gfp-luc2 marker using a lentiviral vector system in a transduction process;
inoculating said xenografts into an animal model prior to selection of transduced cells;
harvesting the xenograft established from the inoculated cells for selection and re-inoculating the selected cells into an animal model; and
harvesting cells from xenograft established from the selected re-inoculated cells for further propagation, analysis or for adaptation into 2D and 3D culture.

22. The method as claimed in claim 21, wherein the transduction of lentiviral vector system includes a combination of hexadimethrine bromide and spinoculation with viral supernatant concentrate.

23. An assay to evaluate the efficacy of an agent against nasopharyngeal cancer (NPC) by measuring the viability of luciferase expressing cells in a 2D and 3D co- or multiple-culture system wherein the assay is a non-lytic luciferase assay and the luciferase expressing cells are NPC patient derived xenograft cells labelled with a gfp-luc2 marker.

24. The assay as claimed in claim 23, wherein the agent is selected from one of the following: drug, bioactive compound, chemical entity, biological agent, irradiation, or a combination thereof.
